(19) United States
(12) Patent Application Publication
(10) Pub. No.: US 2011/0098639 A1
(43) Pub. Date: Apr. 28, 2011

(54) Milk extraction device

(76) Inventor: Claudia Kirchner, Markgroningen (DE)

(21) Appl. No.: 11/630,870

(22) PCT Filed: Jun. 4, 2005
(86) PCT No.: PCT/EP2005/006028

§ 371 Date: Jul. 10, 2007

(30) Foreign Application Priority Data
Jun. 24, 2004 (DE) 10 2004 030 692.3

(51) Int. Cl. A61M 1/06

(52) U.S. Cl. 604/74

(57) ABSTRACT

A milk extraction device including at least one extraction part with an intermediate piece that is, or can be, detachably fastened on the opening of a receptacle and that is linked with a breast piece in a vacuum-tight manner so as to exert a sucking effect on the breast. The device also includes a cap that is, or can be, linked with the intermediate piece in a vacuum-tight manner. An elastic, liquid-tight separating membrane is inserted in the cap and transmits a varying vacuum from the pump unit to the interior of the intermediate piece and the breast piece. The cap is detachably connected to an adapted coupling section of the intermediate piece by a coupling counter-section, thereby meeting increased hygienic standards and simplifying the use of the milk extraction device.

### BACKGROUND OF THE INVENTION

1. Field of the Invention

This invention relates to a milk extraction device, including at least one extraction element having an intermediate piece, which is or can be releasably attached to an opening of a receptacle and is connected in a vacuum-tight manner with a breast attachment element for exerting an extraction action on the breast, as well as a headpiece, which is or can be connected with the attachment element in a vacuum-tight manner and into which an elastic liquid-proof separating membrane is inserted, which transmits a varying vacuum from a pump unit to an interior of the intermediate piece and of the breast attachment element.

2. Discussion of Related Art

A milk extraction device for pumping milk from a human breast is shown in German Patent Reference DE 690 00 542 T2. In this known milk extraction device an intermediate piece, which is connected in one piece with a funnel-shaped breast attachment element, is attached to the opening of a receptacle for milk, so that it can be screwed off. With a portion which is at the top in the position of use it is connected in one piece with a cylinder, which is formed on it and is open toward the top, inside of which a piston for pumping by hand is seated so that it can be moved back and forth. Also, a flexible bellows made of an elastomeric material, such as silicon caoutchouc, for example, is provided, which can be inserted into the cylinder when the piston is removed. With a flexible hose, the bellows is connected to an outside source of an oscillating vacuum, in particular an electric breast pump. The piston, or the bellows must be removed from the cylinder for refitting and cleaning, while the cylinder remains permanently connected with the intermediate piece. In this construction, the cylinder is used for manual pumping, as well as for automatic operation by a pumping unit, and handling while cleaning and refitting is correspondingly connected.

A milk extraction device for human milk is disclosed in European Patent Reference EP 0 123 269 B1, where a movable separating membrane is stretched in a pump housing between a movable piston and a lid and defines a suction chamber together with the lid and can be moved in the suction rhythm. Here, the space with the membrane is arranged in a pump unit, which can result in difficulties during cleaning.

A milk extraction device with a manually operable pumping unit is disclosed in PCT International Application WO 01/34226 A1, while a milk extraction device with an automatic pump unit is disclosed in German Patent Reference DE 102 28 455 A1.

### SUMMARY OF THE INVENTION

The object of this invention is to provide a milk extraction device of the type mentioned above but which has high sanitary requirements that can be satisfied, along with easier manipulation.

This object is achieved with a milk extraction device having the characteristics in this specification and in the claims. The headpiece is releasably connected to a coupling section of the intermediate piece matched to it by a counter-coupling section.

With these steps the separating membrane with the attachment can be removed from the intermediate piece and can be cleaned, or can be easily provided with another headpiece.

Thus, advantageous steps for the construction and manipulation include the coupling section being formed on the intermediate piece, and the counter-coupling section on the headpiece, each respectively in one piece, and embodied vacuum-tight and releasably plugged into each other.

Furthermore, with a dependable coupling of the headpiece and the intermediate piece, the operation is aided by a plug-in connection between the coupling section and the counter-coupling section secured by a rotating connection or a snap-in connection between the intermediate piece and the headpiece.

The function and manipulation of the separating membrane are aided because in the not-charged state of rest the separating membrane is shaped as hollow toward the bottom in relation to the set-up receptacle and is releasably pressed with an upper, circumferential sealing edge against an upper, matched counter-edge of the headpiece.

In this embodiment, the counter-edge of the headpiece is formed on a connecting element, which is open toward the top, against which a cover is releasably pressed with the interposition of a sealing edge. The cover is releasably fixed in place on the connecting element by a snap-in connection or a rotatable locking device.

Steps, wherein the snap-in connection between the intermediate piece and the headpiece is formed by a holding piece in the shape of a resilient snap-in finger at the headpiece and a holding element on the intermediate piece form a simple operation and a secure coupling with each other. The snap-in connection is laid out so that it assumes the snapped-in position when the coupling section and the counter-coupling section are plugged together in their sealing position.

A construction which is advantageous for receiving the separating membrane and manipulating the headpiece is achieved with a hollow space surrounding the separating membrane laterally and on an underside which is partitioned off in the headpiece and is provided on the underside with the counter-coupling section.

Manipulation is favored because in its state where it is fixed on the intermediate piece, the headpiece is provided laterally with respect to the longitudinal extension of the breast attachment element and the transition piece with at least one support section, by which the headpiece with the coupled intermediate piece and connected receptacle can be attached to a matching support element of a housing of the pump unit.

Furthermore, the steps contribute to the dependable functioning of the milk extraction device wherein an inlet valve is attached to a milk outlet section of the intermediate piece to the receptacle, which valve is hollow toward the bottom and has a slit opening, which opens because of the weight of the pumped-out milk flowing through the intermediate piece. In the state of rest, and particularly through the creation of a vacuum in the intermediate piece, the opening is closed because of its elastic embodiment.

The steps wherein the pump unit is connected with the headpiece in a vacuum-tight and releasable manner by a line further contribute to a simple construction and a simple manipulation.

An advantageous operation of the milk extraction device results if two extraction elements of the construction are connected by respective lines to the pump unit, wherein the pump unit has two connectors on a connection section which can be charged with a vacuum in an anti-cyclic manner and to which the lines are or can be connected.

In this case dependable functioning is aided because the pump unit is constructed so that during the vacuum-charging phases the extraction elements can be charged with pressure in an anti-cyclic manner via the connectors.

A further advantage for use is that at least one manual pump headpiece is provided which, corresponding to the headpiece with a formed-on coupling section and elements of a rotatory connection or snap-in connection for securing on the intermediate piece, is equipped in the same way as the headpiece.

### BRIEF DESCRIPTION OF THE DRAWINGS

This invention is explained in greater detail in view of exemplary embodiments, making reference to the drawings, wherein:

FIG. 1 shows a milk extraction device with an automatic pump unit and two extraction elements connected thereto, having respective receptacles and breast connection pieces, in a perspective plan view;

FIG. 2 shows a headpiece of an extraction element, in a perspective view;

FIG. 3 shows a hand pump headpiece, in a lateral view;

FIG. 4 shows an extraction element of the milk extraction device, in a lateral view;

FIG. 5 shows the headpiece in accordance with FIG. 2, in an exploded lateral view;

FIG. 6 shows the headpiece in accordance with FIG. 5, in an exploded perspective view;

FIG. 7 shows a receptacle, in a perspective view;

FIG. 8 shows an intermediate piece with an element of an extraction element, in a perspective view;

FIG. 9 shows a receptacle and an intermediate piece with an extraction element in a separate representation, in a perspective view;

FIG. 10 shows the hand pump headpiece in accordance with FIG. 3, in an exploded perspective view; and

FIG. 11 shows an extraction element with a hand pump headpiece, in a lateral view.

### DETAILED DESCRIPTION OF THE INVENTION

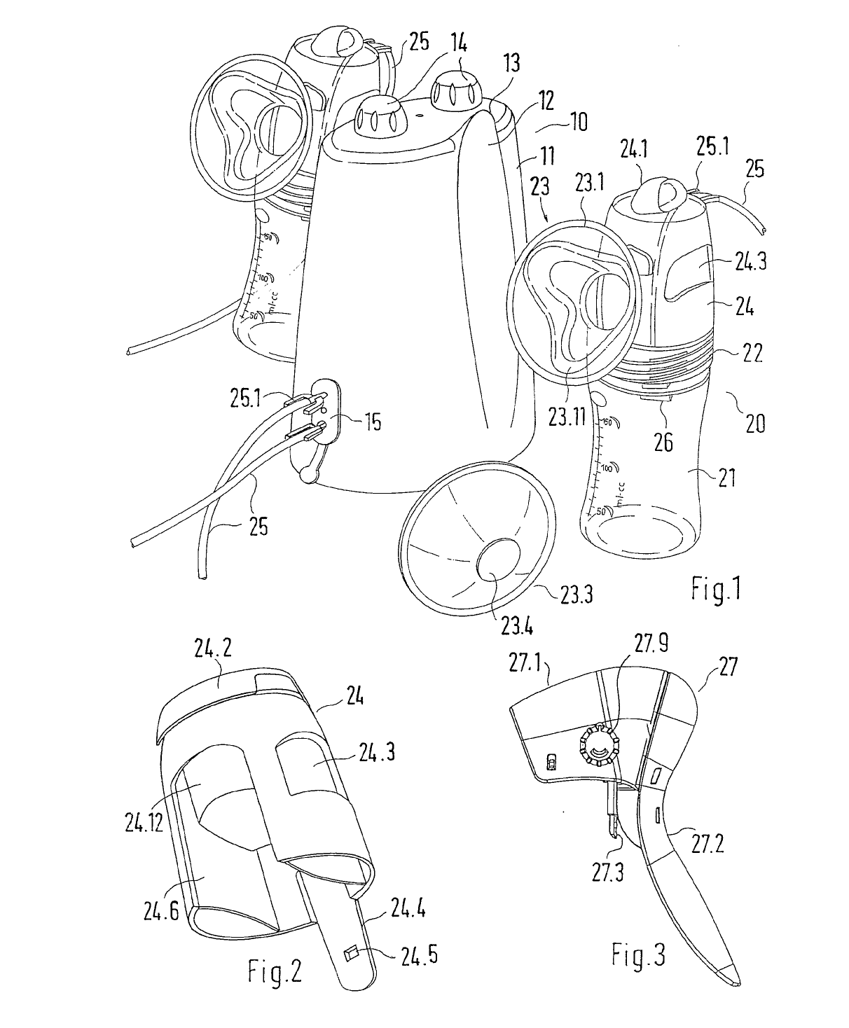

A milk extraction device for human milk, having an automatic, electrically operated pump unit 10 and two extraction elements, which are connected via respective lines 25 to a connecting section 15 of a housing 11 of the pump unit 10, is shown in FIG. 1. As essential components, the respective extraction elements respectively have a receptacle 21, for example in the form of a bottle or a bag for receiving the pumped-out milk, an intermediate piece 22 attached, in particular screwed, to its opening, with an extraction element 23 attached in one piece and a headpiece 24 pushed on the intermediate piece 22, to which the end of the respective line 25 facing away from the pump unit 10 is connected, in order to generate a changing vacuum in the interior of the intermediate piece 22 and the extraction element 23 for extracting the milk. Adjusting elements 14 are arranged on the housing 11 of the pump unit 10 to set the strength of the vacuum and the length of the periods of the extraction cycles and, if required, the chronological course of the strength of the vacuum or of the wave shape.

The lines 25 have connecting elements 25.1 on one or both ends, by which they can be easily and sealingly connected by one end to the two connectors of the connecting section 15 on the housing 11, and by the other end to the headpiece 24, by simply plugging them in, and on a front section facing the breast, the extraction element 23 has a funnel-shaped breast attachment element 23.1 with a central opening on the outlet side, which makes a transition into a transition element 23.2 formed in one piece on the intermediate piece 22. A flexible extraction insert 23.3 is inserted into the funnel-shaped breast attachment element 23.1 and, with an edge section, extends past or beyond the free edge of the breast attachment element 23.1 and is maintained on it in a vacuum-tight manner. For aiding the extraction action, the flexible extraction insert 23.3, for example made of silicon caoutchouc, is seated elastically and resiliently by spacing structures, for example nubs on a rear and groove-like structures, on the facing outside of the per se relatively hard breast attachment element 23.1 in the inner area of the funnel-shaped breast attachment element 23.1. The space between the extraction insert 23.3 and the inside of the breast attachment element 23.1 is connected via the groove structure with the interior of the intermediate piece 22 and furthermore with the pump unit 10 for transmitting the vacuum. The extracted milk enters the interior of the intermediate piece 22 via a central opening 23.4 of the extraction insert 23.3 and via an inlet valve 26, also see FIG. 9, arranged on the intermediate piece 22 at the inlet to the receptacle 21, into the milk reception area of the receptacle 21.

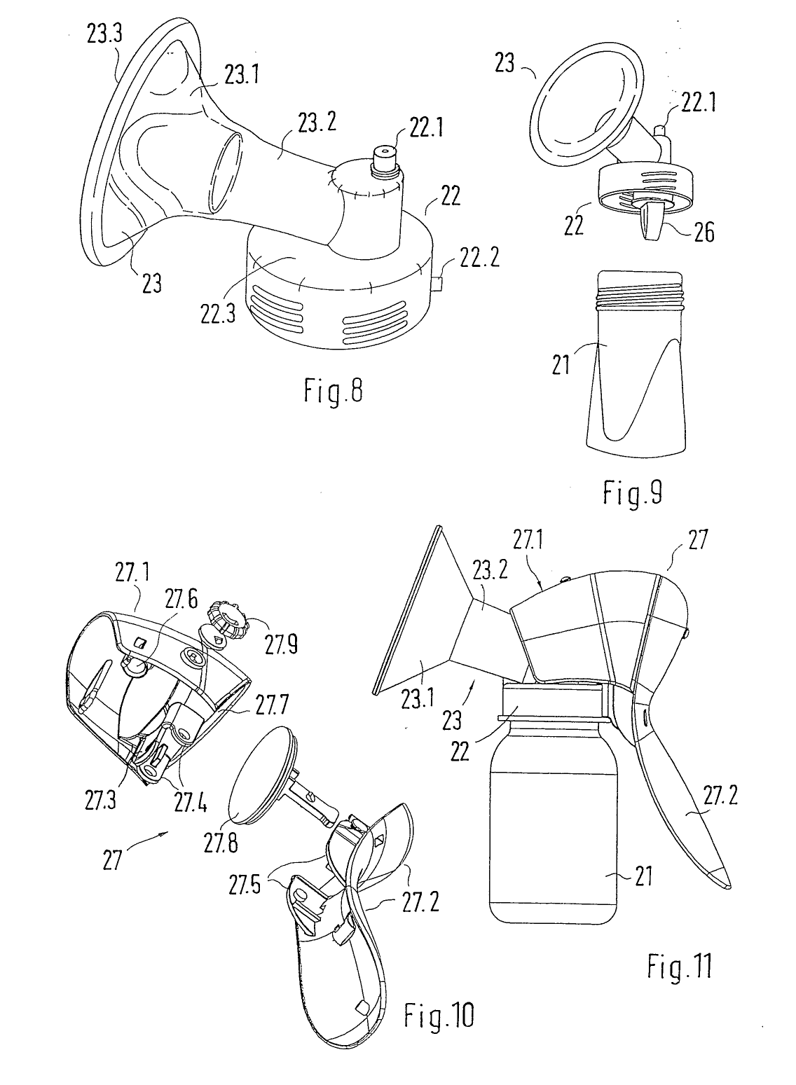

The headpiece 24 extends in the form of a cap over the upper section of the intermediate piece 22, and its counter-coupling section 24.11, which is matched to the coupling section, is pushed in a vacuum-tight manner onto the nozzle-shaped coupling section 22.1 formed on the top of the intermediate piece 22 for transmitting the vacuum from the pump unit 10 to the interior of the intermediate piece 22 and the extraction element 23, see also FIGS. 6, 8 and 9. Here, the counter-coupling section 24.11 extends over the groove-like coupling section 22.1 or, vice versa, the groove-like coupling section 22.1 extends over the counter-coupling section 24.11. A hollow through-groove is formed between the interior of the intermediate piece 22 and an inner chamber of the headpiece 24. For securing the plugged-in connection between the headpiece 24 and the intermediate piece 22, a holding element 22.2, protruding toward the rear or cut in, is formed on a rear section of the intermediate piece 22 facing away from the extraction element 23, preferably in the area of the outer circumference of the screw section which, in the completely plugged-in headpiece 24, engages a holding cut 24.5 or holding protrusion of a resilient, finger-like holding piece 24.4, which is formed in one piece on a lower edge area of the headpiece 24 and which can be brought out of engagement with the protruding holding element 22.2 for releasing the snap-in connection thus formed, as can be seen, for example, in FIGS. 2, 4, 5, 6 and 8.

Laterally with respect to an imagined vertical longitudinal center plane from the front to the rear of the extraction element 20, the headpiece 24 has on both sides support sections 24.3, by which it can be suspended in an upper, matching support element 13 formed on the housing 11. The support section 24.3 is embodied as a recess in the respective wall section of the headpiece 24, as shown in FIGS. 1, 3 and 6. Also, below the support elements 13, which extend upward in the shape of ears, the housing 11, which is oval in cross section and tapers toward the top, has bearing sections 12, which in cross section are concave toward an inside and extend in the longitudinal direction of the housing 11, into which the curves of the suspended extraction elements 20, which are convex toward the outside, easily fit, so that a certain support on the housing 11 is provided in the suspended state of the extraction elements 20, but on the other hand the extraction elements 20 can be easily removed. For extending over the intermediate piece 22, the headpiece 24 has on its front a recess 24.6, open toward the bottom, in its outer wall, through which, in the state in which the headpiece is placed on top, the transition element 23.2 extends, which is shown in FIGS. 2, 4 and 6, in particular.

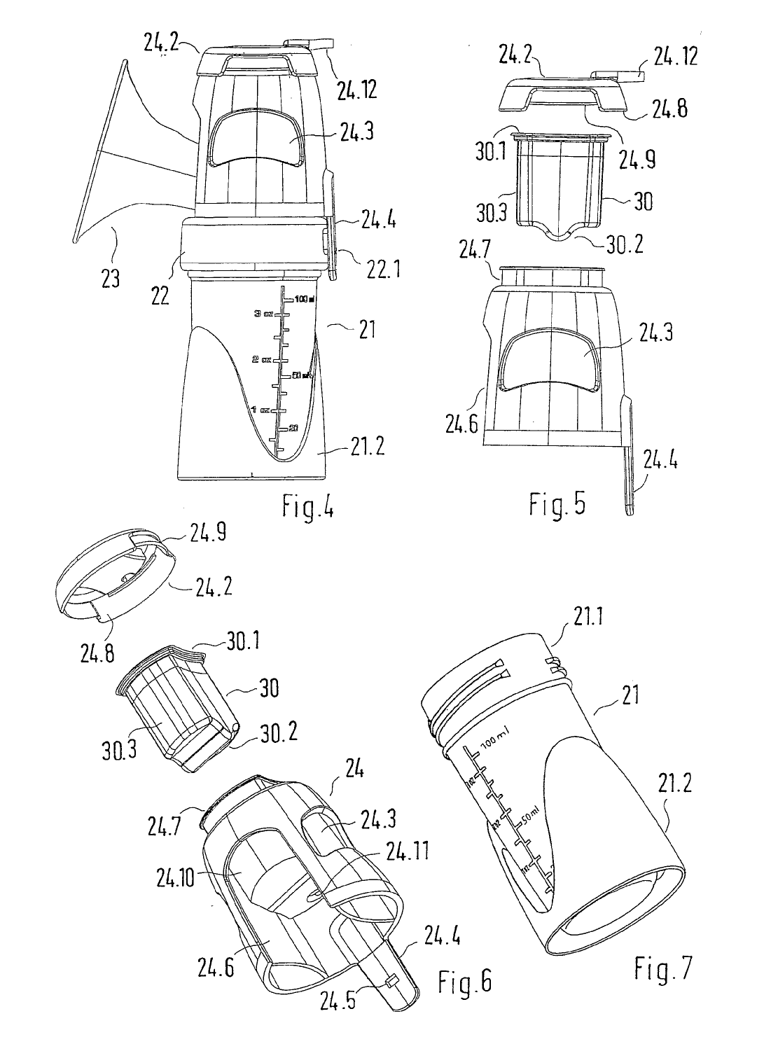

A hollow space 24.10, open toward the top, is formed on the inside of the headpiece 24, into which bottom, that extends approximately half upward, the counter-coupling section 24.11 is cut, as FIG. 6 shows. The hollow space 24.10 is surrounded in the longitudinal direction oriented from the bottom to the top by an outer wall, which makes a transition into an upwardly protruding connecting element 24.7 having an upper edge, such as shown in FIGS. 6 and 7, for example. A separating membrane 30, hollow toward the bottom, is removably seated in the hollow space 24.10, whose upper sealing edge 30.1 rests sealingly against the upper edge of the connecting element 24.7 and is pressed against it by a cover 24.2 in the form of a lid-like element. For pressing it down, the cover or cover means 24.2 has an inner cover edge 24.9, and an outer cover edge 24.8 for at least partially extending past or beyond the connecting element 24.7, which has a snap-in structure on the inside in order to fix the cover means 24.2 in place on the connecting element 24.7. For example, in this case the snap-in structure is embodied so that it can be easily released by pressing the cover means 24.2 inward on both sides, which results in its becoming oval. A passage on a connecting element 24.12 terminates on the inside of the cover means 24.2, to which the appropriate end section of the line 25 can be connected, such as shown for example, in FIGS. 4, 5 and 6.

As FIG. 1 shows, an insertion piece 24.1 can be inserted into the top of the headpiece 24 in place of the cover means 24.2 for example, into which the appropriate line 25 is inserted with the connecting element 25.1, while the connecting element 25.1 is not needed when the cover means 24.2 with the connecting element 24.12 is employed.

The separating membrane 30 introduced into the hollow space 24.10 forms the liquid-tight separation in the hollow vacuum space between the pump unit 10 and the interior of the intermediate piece 22 and the extraction element 23, so that the entry of milk into the lines 25 is dependably prevented. The separating membrane 30 is made of a flexible, soft material, in particular a silicon material, for transmitting the vacuum from the pump unit 10 to the extraction element 23. The downward projecting hollow space of the separating membrane 30 is surrounded on the bottom by a membrane bottom 30.2, and laterally by a circumferential membrane wall 30.3, and is open on its top. For improving its function, a downward bow is formed in the center area of the membrane bottom 30.2, as shown in greater detail in FIGS. 5 and 6. The bow is closed off on two sides by an extension of two membrane wall sections, which are facing each other and are flat, or arch slightly into the hollow space of the separating membrane 30. Otherwise, the membrane wall 30.3 between these lateral sections is embodied in cross section so that it arches outward toward the upper edge and has an approximately oval cross section.

The inlet valve 26 is sealingly placed on a nozzle-shaped section on the underside of the intermediate piece 22 and projects, tapering downward in cross section, into the opening of the receptacle 21 in a wedge-shaped manner, wherein the underside of the inlet valve 26 has a slit, formed by lateral walls touching each other which, when milk flows in, opens because of the weight of the latter, but closes in the position of rest and in particular when a vacuum appears, as a result of the elasticity in the slit area of bordering walls of the inlet valve 26, so that a vacuum is dependably formed in the extraction chamber of the extraction element 23. For securely preventing the sticking together of the ends of the inlet valve 26 forming the slit, the pump unit 10 is such that, after being charged with a vacuum, it exerts an overpressure on the lines 25 via a rotary valve arranged in its interior, as explained in greater detail in the German Patent Publication DE 102 28 455 A1 mentioned at the outset. Thus it is possible to embody the inlet valve 26 so it dependably closes in the area of the slit, wherein opening and flow of the milk into the receptacle 21 is assured by the charge with overpressure. Advantageously, the intermediate piece 22 has a vent opening 22.3 in an upper area of the screw section, by which the interior of the receptacle 21 is vented.

The automatic pump unit 10, which is preferably electrically operated, is constructed so that a vacuum is alternatingly, or anti-cyclically, generated at both connections of the connecting section 15 and is transmitted via the lines 25 to the respective extraction elements 20. The above mentioned overpressure generation at the two connections of the connecting section 15 can also be caused anti-cyclically between the phases of vacuum generation, so that the inlet valves 26 can be dependably opened and the extraction action at the breast can also be improved. Further setting options of the extraction action are offered via the adjusting elements 14 and, if required, further adjusting elements, for example for regulating secondary air.

The milk extraction device is designed so that a manual pump headpiece 22 in accordance with FIGS. 3, 10 and 11, corresponding to the headpiece 24, can be placed with a few movements on the coupling section 22.1 and the holding element 22.2 of the intermediate piece 22. For this purpose, the manual pump headpiece 22 has a counter-coupling section 27.6 arranged on an underside, which is coupled to the coupling section 22.1, and on its rear facing away from the extraction element 23 has a holding element 27.3 in the form of a resilient snap-in finger with a holding engagement piece, so that the plug-in connection between the coupling section 22.1 and the counter-coupling section 27.6 is assured by the snap-in finger snapping into the holding element 22.2, such as described in connection with the headpiece 24. It is also possible to provide rotary securing, for example with two projections engaging each other by rotation. As described in greater detail in PCT International Application WO 01/34226 A1 mentioned above, the manual pump headpiece 27 has a cap section 27.1 which is convexly arched toward the top, underneath which a curved pump cylinder is formed as the piston chamber 27.7 for a piston 27.8 which can be moved back and forth by a manual actuating section 27.2. On its side, the piston chamber 27.2 has a secondary air regulating unit 27.9 leading to the atmosphere, by which the extraction action can be regulated. For moving the piston 27.8, the actuating section 27.2 is pivotably seated underneath the cap section 27.1 in lateral pivot bearings 27.4 by bearing sections 27.5, wherein one direction of movement takes place by a spring force and the other direction of movement manually against the spring force. It is also possible to place a fixed piston section with a counter-coupling section 27.6 on the coupling section 22.1 of the intermediate piece 22, by which a piston chamber can be moved back and forth, which is movable by a manual actuating section, wherein otherwise the embodiment of such a manual pump headpiece can be designed similar to that as described above.

FIGS. 4 and 7 show an exemplary embodiment of a receptacle 21 in which a support section 21.2, which widens toward the bottom is attached underneath a connecting section 21.1 in the opening area for secure placement, because of which the operation is also eased.

The described milk extraction device provides a simple operation because of easy handling when connecting headpieces 24, or manual headpieces 27, during which increased sanitary requirements are satisfied.

1. A milk extraction device, comprising at least one extraction element (20) having an intermediate piece (22) releasably attached to an opening of a receptacle (21) and connected in a vacuum-tight manner with a breast attachment element (23.1) for exerting an extraction action on a breast, a headpiece (24) connectible with the attachment element in a vacuum-tight manner and into which an elastic liquid-proof separating membrane (30) is inserted, which transmits a variable vacuum from a pump unit (10) to an interior of the intermediate piece (22) and of the breast attachment element (23.1), wherein
the headpiece (24) is releasably connected to a coupling section (22.1) of the intermediate piece (22) by a matched counter-coupling section (24.11).

2. The milk extraction device in accordance with claim 1, wherein the coupling section (22.1) is formed on the intermediate piece (22), the counter-coupling section (24.11) is formed on the headpiece (24), each respectively in one piece and vacuum-tight and plugged into each other.

3. The milk extraction device in accordance with claim 2, wherein the plug-in connection between the coupling section (22.1) and the counter-coupling section (24.11) is secured by one of a rotating connection and a snap-in connection between the intermediate piece (22) and the headpiece (24).

4. The milk extraction device in accordance with claim 3, wherein in a not-charged state of rest, the separating membrane (30) is shaped hollow toward a bottom with respect to a set-up receptacle (21) and releasably pressed with an upper, circumferential sealing edge (30.1) against an upper matched counter-edge of the headpiece (24).

5. The milk extraction device in accordance with claim 4, wherein the counter-edge of the headpiece (24) is formed on a connecting element (24.7) open toward a top, against which a cover (24.2) is releasably pressed with an interposition of the sealing edge (30.1).

6. The milk extraction device in accordance with claim 5, wherein cover (24.2) is releasably fixed in place on the connecting element (24.7) by one of a snap-in connection and a rotatable locking device.

7. The milk extraction device in accordance with claim 6, wherein the snap-in connection between the intermediate piece (22) and the headpiece (24) is formed by a holding piece (24.4) in a shape of a resilient snap-in finger at the headpiece (24) and a holding element (22.2) on the intermediate piece (22), wherein the snap-in connection assumes a snapped-in position when the coupling section (22.1) and the counter-coupling section (24.11) are plugged together in a sealing position.

8. The milk extraction device in accordance with claim 7, wherein a hollow space (24.10) surrounding the separating membrane (30) laterally and on an underside is partitioned off in the headpiece (24) and has on the underside the counter-coupling section (24.11).

9. The milk extraction device in accordance with claim 8, wherein when fixed on the intermediate piece (22) the headpiece (24) has laterally with respect to the longitudinal extension of the breast attachment element (23.1) and the transition piece (23.2) at least one support section (24.3) by which the headpiece (24) with the coupled intermediate piece (22) and connected receptacle (21) can be attached to a matching support element (13) of a housing (11) of the pump unit (10).

10. The milk extraction device in accordance with claim 9, wherein an inlet valve (26) is attached to a milk outlet section of the intermediate piece (22) to the receptacle (21), the inlet valve is hollow toward the bottom and has a slit opening which opens due to a weight of the pumped-out milk flowing through the intermediate piece (22) which closes when a vacuum is created in the intermediate piece (22).

11. The milk extraction device in accordance with claim 10, wherein the pump unit (10) is connected with the headpiece (24) in a vacuum-tight and releasable manner by a line (25).

12. The milk extraction device in accordance with claim 11, wherein two extraction elements (20) are connected by respective lines (25) to the pump unit (10), and the pump unit (10) has two connectors on a connection section (15) which can be charged with a vacuum in an anti-cyclic manner and to which the lines are connectible.

13. The milk extraction device in accordance with claim 12, wherein the pump unit (10) during vacuum-charging phases charges the extraction elements with pressure in an anti-cyclic manner via the connectors.

14. The milk extraction device in accordance with claim 13, wherein at least one manual pump headpiece (27) corresponding to the headpiece (24) with a formed-on coupling section (24.11) and elements of one of a rotatory connection and a snap-in connection for securing on the intermediate piece (22), is equipped in a same way as the headpiece (24).

15. The milk extraction device in accordance with claim 1, wherein in a not-charged state of rest, the separating membrane (30) is shaped hollow toward a bottom with respect to a set-up receptacle (21) and releasably pressed with an upper, circumferential sealing edge (30.1) against an upper matched counter-edge of the headpiece (24).

16. The milk extraction device in accordance with claim 15, wherein the counter-edge of the headpiece (24) is formed on a connecting element (24.7) open toward a top, against which a cover (24.2) is releasably pressed with an interposition of the sealing edge (30.1).

17. The milk extraction device in accordance with claim 16, wherein the cover (24.2) is releasably fixed in place on the connecting element (24.7) by one of a snap-in connection and a rotatable locking device.

18. The milk extraction device in accordance with claim 3, wherein the snap-in connection between the intermediate piece (22) and the headpiece (24) is formed by a holding piece (24.4) in a shape of a resilient snap-in finger at the headpiece (24) and a holding element (22.2) on the intermediate piece (22), wherein the snap-in connection assumes a snapped-in position when the coupling section (22.1) and the counter-coupling section (24.11) are plugged together in a sealing position.

19. The milk extraction device in accordance with claim 1, wherein a hollow space (24.10) surrounding the separating membrane (30) laterally and on an underside is partitioned off in the headpiece (24) and has on the underside the counter-coupling section (24.11).

20. The milk extraction device in accordance with claim 1, wherein when fixed on the intermediate piece (22) the headpiece (24) has laterally with respect to the longitudinal extension of the breast attachment element (23.1) and the transition piece (23.2) at least one support section (24.3) by which the headpiece (24) with the coupled intermediate piece (22) and connected receptacle (21) can be attached to a matching support element (13) of a housing (11) of the pump unit (10).

21. The milk extraction device in accordance with claim 1, wherein an inlet valve (26) is attached to a milk outlet section of the intermediate piece (22) to the receptacle (21), the inlet valve is hollow toward the bottom and has a slit opening which opens due to a weight of the pumped-out milk flowing through the intermediate piece (22) which closes when a vacuum is created in the intermediate piece (22).

22. The milk extraction device in accordance with claim 1, wherein the pump unit (10) is connected with the headpiece (24) in a vacuum-tight and releasable manner by a line (25).

23. The milk extraction device in accordance with claim 1, wherein two extraction elements (20) are connected by respective lines (25) to the pump unit (10), and the pump unit (10) has two connectors on a connection section (15) which can be charged with a vacuum in an anti-cyclic manner and to which the lines are connectible.

24. The milk extraction device in accordance with claim 23 wherein the pump unit (10) during vacuum-charging phases charges the extraction elements with pressure in an anti-cyclic manner via the connectors.

25. The milk extraction device in accordance with claim 3, wherein at least one manual pump headpiece (27) corresponding to the headpiece (24) with a formed-on coupling section (24.11) and elements of one of a rotatory connection and a snap-in connection for securing on the intermediate piece (22), is equipped in a same way as the headpiece (24).
